(19) United States
(12) Patent Application Publication
(10) Pub. No.: US 2026/0184695 A1
(43) Pub. Date: Jul. 2, 2026

(54) PROCESSES AND INTERMEDIATES FOR THE PREPARATION OF A PYRIMIDINE AMINOPYRAZOLE COMPOUND

(71) Applicant: DENALI THERAPEUTICS INC., South San Francisco, CA (US)

(72) Inventors: Katrien Brak, Belmont, CA (US); Trevor C. Johnson, South San Francisco, CA (US); Travis Remarchuk, South San Francisco, CA (US); Anantha Sudhakar, South San Francisco, CA (US); Jeffrey Deignan, San Francisco, CA (US)

(21) Appl. No.: 19/131,683

(22) PCT Filed: Nov. 21, 2023
(86) PCT No.: PCT/US2023/080675

§ 371 Date: May 21, 2025

Related U.S. Application Data
(60) Provisional application No. 63/427,303, filed on Nov. 22, 2022.

(51) Int. Cl. C07D 403/14; C07D 239/30; C07D 239/34; C07D 249/04; C07D 403/06; C07F 7/08

(52) U.S. Cl. CPC C07D 403/14; C07D 239/30; C07D 239/34; C07D 249/04; C07D 403/06; C07F 7/0814; C07F 7/083

(57) ABSTRACT

The present disclosure relates to methods of making N²-(3-(2-(2H-1, 2, 3-triazol-2-yl)propan-2-yl)-1-cyclopropyl-1H-pyrazol-5-yl)-N⁴-ethyl-5-(trifluoromethyl)pyrimidine-2,4-diamine and intermediates thereof.

### CROSS REFERENCE TO RELATED APPLICATION

This application claims the benefit under 35 U.S.C. § 119(e) to U.S. Provisional Application No. 63/427,303, filed Nov. 22, 2022, which is incorporated by reference in its entirety.

### FIELD

The present disclosure relates to methods of making a pyrimidine aminopyrazole compound and intermediates thereof. The compound is an inhibitor of LRRK2 kinase and find use for treatment of LRRK2-mediated diseases such as Parkinson's disease.

### DESCRIPTION

Leucine-rich repeat kinase 2 (LRRK2) is a complex signaling protein that is a key therapeutic target, particularly in Parkinson's disease (PD). Combined genetic and biochemical evidence implicates certain kinase function in the pathogenesis of neurodegenerative disorders (Christensen, K. V. (2017) Progress in medicinal chemistry 56:37-80; Fuji, R. N. et al (2015) Science Translational Medicine 7(273):273ra15; Taymans, J. M. et al (2016) Current Neuropharmacology 14(3):214-225). Kinase inhibitors are under investigation for treatment of Alzheimer's disease, Parkinson's disease, ALS and other diseases (Estrada, A. A. et al (2015) J. Med. Chem. 58(17): 6733-6746; Estrada, A. A. et al (2013) J. Med. Chem. 57:921-936; Chen, H. et al (2012) J. Med. Chem. 55:5536-5545; Estrada, A. A. et al (2015) J. Med. Chem. 58:6733-6746; Chan, B. K. et al (2013) ACS Med. Chem. Lett. 4:85-90; WO2017218843; U.S. Pat. Nos. 8,354,420; 8,569,281; 8,791,130; 8,796,296; 8,802,674; 8,809,331; 8,815,882; 9,145,402; 9,212,173; 9,212,186; 9,932,325, U.S. Ser. No. 10/590,114, U.S. Ser. No. 11/111,235, and WO 2012/062783.

The present disclosure relates to methods of making a LRRK2 inhibitor N²-(3-(2-(2H-1,2,3-triazol-2-yl)propan-2-yl)-1-cyclopropyl-1H-pyrazol-5-yl)-N⁴-ethyl-5-(trifluoromethyl)pyrimidine-2,4-diamine and intermediates thereof, the inhibitor also referred to herein as the Formula I compound and having the structure:

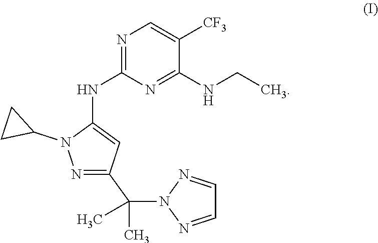 [SMILES: CCNc1nc(Nc2cc(C(C)(C)n3nccn3)nn2C2CC2)ncc1C(F)(F)F]

In one aspect, provided is a method for preparing compound I or a salt thereof

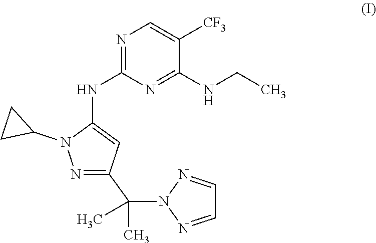 [SMILES: CCNc1nc(Nc2cc(C(C)(C)n3nccn3)nn2C2CC2)ncc1C(F)(F)F]

comprising:
a) contacting compound II with a compound Formula B and a base to produce a compound of Formula C

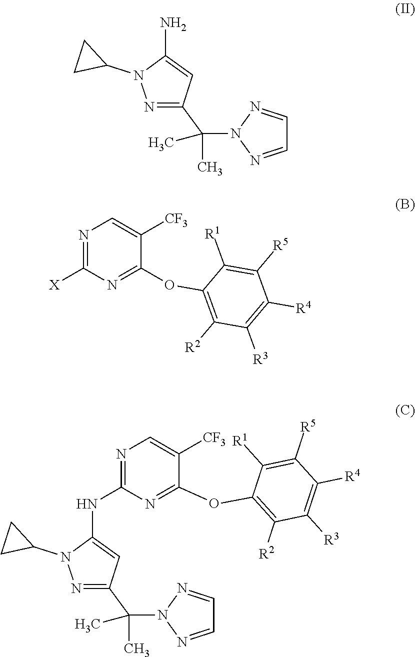

wherein R¹, R², R³, R⁴, and R⁵ are independently H, cyano, halo, methyl, or NO₂; and
b) contacting a compound of Formula C with ethylamine;
   under conditions sufficient to produce compound I.

In some embodiments, the compound of Formula B is compound B-1

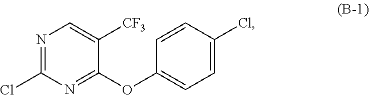 [SMILES: FC(F)(F)c1cnc(Cl)nc1Oc1ccc(Cl)cc1]

and the compound of Formula C is compound C-1

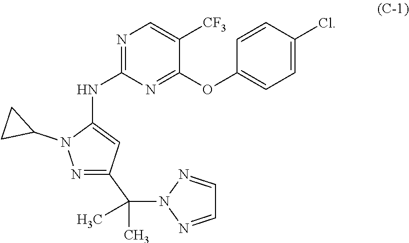 [SMILES: CC(C)(c1cc(Nc2ncc(C(F)(F)F)c(Oc3ccc(Cl)cc3)n2)n(C2CC2)n1)n1nccn1]

In some embodiments, provided is B-1 in 90% or greater regioisomeric purity. In other embodiments, provided is B-1 in 95% or greater regioisomeric purity. In some embodiments, provided is B-1 in 96% or greater regioisomeric purity. In other embodiments, provided is B-1 in 97% or greater regioisomeric purity. In still other embodiments provided is B-1 in 98% or greater regioisomeric purity. In some embodiments, provided is compound B-1 in 99% or greater regioisomeric purity. The regioisomer of B-1 is meant to be a compound having the structure:

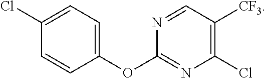 [SMILES: FC(F)(F)c1cnc(Oc2ccc(Cl)cc2)nc1Cl]

In some embodiments, provided is C-1 in 96% or greater regioisomeric purity. In other embodiments, provided is C-1 in 97% or greater regioisomeric purity. In still other embodiments provided is C-1 in 98% or greater regioisomeric purity. In some embodiments, the compound C-1 is produced in 99% or greater regioisomeric purity. The regioisomer of C-1 is meant to be a compound having the structure:

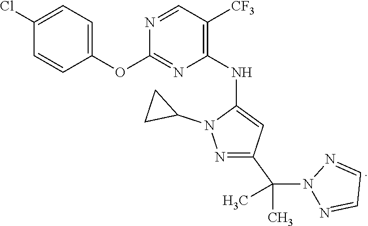 [SMILES: CC(C)(c1cc(Nc2nc(Oc3ccc(Cl)cc3)ncc2C(F)(F)F)n(C2CC2)n1)n1nccn1]

In some embodiments, the base is 2,6-lutidine or 2,4,6-collidine. In some embodiments, the reaction is done in NMP or DMSO. In other embodiments, the reaction is done in DMF or DMAc.

In some embodiments, compound II is contacted with compound B-1 at a temperature from about 60° C. to about 70° C.

In some embodiments, Formula C is contacted with ethylamine in a polar aprotic solvent. In other embodiments the solvent is THF. In still other embodiments the solvent is DMF, DMAc, NMP, and DMSO. In another embodiment the solvent is NMP.

In one aspect, the compound B-1 is prepared by
a) contacting compound D-1 with compound D-2 and a base under conditions sufficient to the produce the compound B-1; and

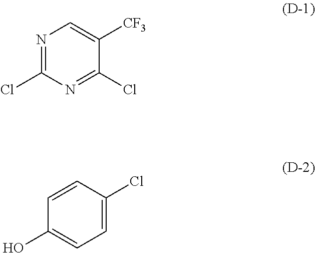 [SMILES: FC(F)(F)c1cnc(Cl)nc1Cl ; Oc1ccc(Cl)cc1]

b) optionally crystallizing the compound B-1 from heptane, isopropanol, or an isopropanol/water mixture.

In some embodiments, the compound B-1 is crystallized from heptane. In some such embodiments, the compound B-1 is crystallized from heptane in 99% or greater regioisomeric purity. In other embodiments, the compound B-1 is crystallized from isopropanol. In some embodiments, compound B-1 is crystallized from isopropanol in 99% or greater regioisomeric purity. In some such embodiments, the compound B-1 is crystallized from an isopropanol/water mixture in 99% or greater regioisomeric purity. In certain embodiments, the isopropanol/water mixture contains at least 50%, 60%, 70%, 80%, or 90% by volume isopropanol.

In some such embodiments, the base is an inorganic base. In some such embodiments, the inorganic base is K₂CO₃ or NaOH. In other embodiments, the base is an organic amine base. In some such embodiments the organic amine base is TEA or DIPEA. In some embodiments the reaction is conducted at a temperature of about 30° C. or less. In other embodiments, the reaction is conducted at 20° C. or less.

In one aspect, the compound D-1 is prepared in situ by contacting compound E-1 with POCl₃

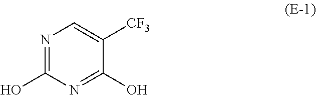 [SMILES: Oc1ncc(C(F)(F)F)c(O)n1]

under conditions sufficient to produce the compound D-1.

In some embodiments, the compound E-1 is contacted with POCl₃ and diisopropylethylamine.

In some embodiments, the compound I is obtained in greater than 98% purity. In some embodiments, the compound I is obtained in greater than 99% purity. In other embodiments, the compound I is obtained in greater than 99.5% purity.

In one aspect, compound II is prepared by contacting compound III with compound IV and an acid

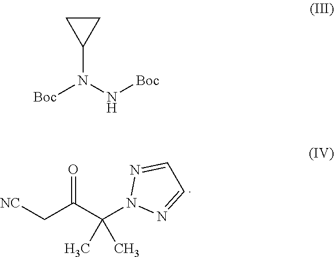 [SMILES: CC(C)(C)OC(=O)NN(C(=O)OC(C)(C)C)C1CC1 ; CC(C)(C(=O)CC#N)n1nccn1]

In some embodiments, the acid is a strong acid. In other embodiments the acid is MSA, BSA, PTSA, HBr, or TFA. In still other embodiments the acid is H₂SO₄ or HCl in an alcoholic solvent. In some such embodiments the alcoholic solvent is i-PrOH, or MeOH. In other embodiments the alcoholic solvent is EtOH.

In some embodiments, compound III is contacted with compound IV at a temperature from about 50° C. to about 60° C.

In another aspect, compound IV is prepared by
a) contacting compound V with a compound of Formula VI and a first base to produce a compound of Formula VII

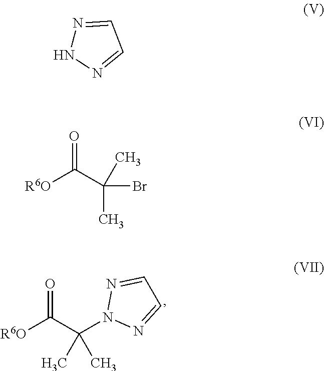

wherein R⁶ is alkyl;
b) washing the compound of Formula VII with an aqueous solution to remove N¹-triazole regioisomer and obtain the compound of Formula VII in 95% or greater regioisomeric purity; and
c) contacting the compound of Formula VII with a second base and CH₃CN; under conditions sufficient to produce the compound IV.

In some embodiments, R⁶ is methyl and the compound of Formula VII is VII-1

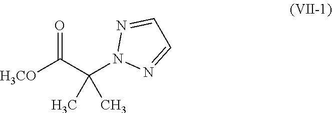

having 95% or greater regioisomeric purity.

In some embodiments, the compound of Formula VII is obtained in 98% or greater regioisomeric purity.

In some embodiments, the compound of Formula VII is washed with an aqueous solution at least twice. In some embodiments, the aqueous solution is water. In other embodiments, the aqueous solution is an acidic aqueous solution. In still other embodiments, the acidic aqueous solution is aqueous HCl.

In some embodiments, the first base is an inorganic base. In some embodiments the base is an alkoxide base. In some such embodiments, the first base is NaOt-Bu. In some embodiments the solvent is THF, CH₃CN, NMP, DMF, or DMAc.

In some embodiments, the second base is n-BuLi, KOt-Bu, LiHMDS, LDA, NaOt-Bu, or KOt-Amyl. In other embodiments, the second base is n-BuLi. In still other embodiments, the second base is LiHMDS.

In some embodiments, the compound IV is obtained in at least 99% or greater purity.

In another aspect, the compound IV is prepared by
a) contacting a compound of formula VIII

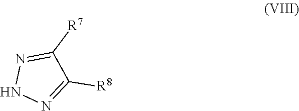

with a compound of Formula VI and a first base to produce a compound of Formula IX

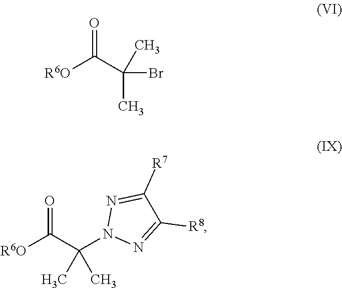

wherein R⁶ is alkyl and R⁷ and R⁸ are independently Br or trimethylsilyl (TMS);
b) contacting the compound of Formula IX when at least one of R⁷ and R⁸ is Br with H₂ or HCO₂H and a hydrogenation catalyst;
and/or contacting the compound of Formula IX when at least one of R⁷ and R⁸ is TMS with base;
to produce a compound of Formula VII

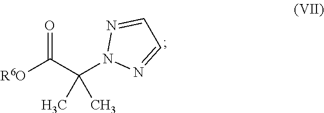

and
c) contacting a compound of Formula VII with a second base and CH₃CN; under conditions sufficient to produce the compound IV.

In some embodiments, R⁶ is CH₃ and R⁷ is Br.

In some embodiments, the hydrogenation catalyst is a Pd catalyst.

In some embodiments, the first base is an inorganic base. In some such embodiments, the first base is K₂CO₃.

In some embodiments, the second base is n-BuLi, KOt-Bu, LiHMDS, LDA, NaOt-Bu or KOtAmyl. In some embodiments, the second base is n-BuLi, KOt-Bu, LiHMDS, or KOtAmyl. In some such embodiments, the second base is n-BuLi. In still other embodiments, the second base is LiHMDS.

In one aspect, provided is a compound of Formula IX

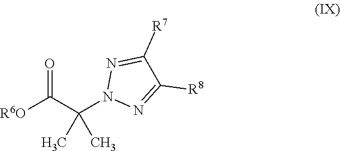

or a salt thereof, wherein:
R⁶ is alkyl and R⁷ and R⁸ are independently Br or trimethylsilyl (TMS).

In one aspect, provided is a compound that is

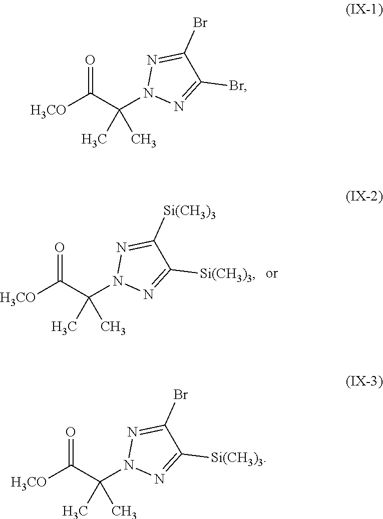

In one aspect, provided is a compound of Formula B:

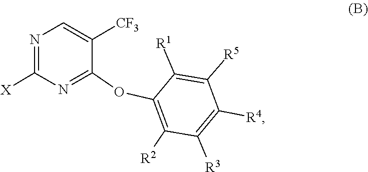

or a salt thereof, wherein:
X is chloro or

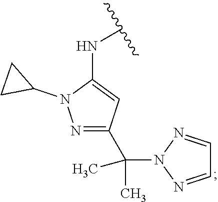 [SMILES: *Nc1cc(C(C)(C)n2nccn2)nn1C1CC1]

and
R¹, R², R³, R⁴, and R⁵ are independently H, cyano, halo, methyl, or NO₂, provided that
a) when X is Cl and R¹, R², R³, and R⁵ are H, then R⁴ is not NO₂ or H;
b) when X is Cl and R¹ and R² are CH₃, then R⁴ is not cyano; and
c) when X is Cl, then R³ is not NO₂.

In one embodiment, provided is a compound that is

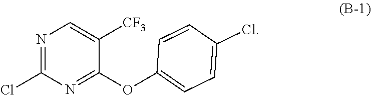 [SMILES: FC(F)(F)c1cnc(Cl)nc1Oc1ccc(Cl)cc1]

or a salt thereof. In another embodiment, provided is a compound that is B-1.

In one embodiment, provided is a compound that is

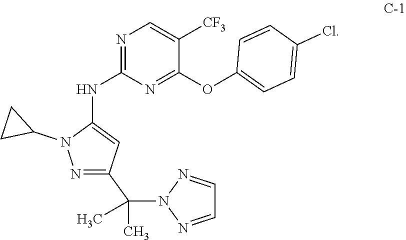 [SMILES: CC(C)(c1cc(Nc2ncc(C(F)(F)F)c(Oc3ccc(Cl)cc3)n2)n(C2CC2)n1)n1nccn1]

### Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

Reference to “about” a value or parameter herein includes and describes embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”.

The term “alkyl” as used herein refers to a saturated linear or branched-chain monovalent hydrocarbon radical of one to twelve carbon atoms (C₁-C₁₂), wherein the alkyl radical may be optionally substituted independently with one or more substituents described below. In another embodiment, an alkyl radical is one to eight carbon atoms (C₁-C₈), or one to six carbon atoms (C₁-C₆). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (amyl, n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, and 1-octyl.

The term salt include, for example, salts with inorganic acids, and salts with an organic acid. Salts may be produced by dissolving the free base in a suitable organic solvent and treating the solution with an acid or with reagents that generate an acid in situ in accordance with conventional procedures for preparing acid addition salts from base compounds. Acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates, and the like. Examples of organic acid addition salts include salts of propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, ethanesulfonic acid, salicylic acid, and the like.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as 3H, 13C and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “reaction conditions” and “sufficient reaction conditions” is intended to refer to the physical and/or environmental conditions under which a chemical reaction proceeds. Examples of reaction conditions include, but are not limited to, one or more of following: reaction temperature, solvent, pH, pressure, reaction time, mole ratio of reactants, the presence of a base or acid, one or more protecting groups, or catalyst, radiation, etc. Reaction conditions may be named after the particular chemical reaction in which the conditions are employed, such as, coupling conditions, hydrogenation conditions, acylation conditions, reduction conditions, etc. Reaction conditions for most reactions are generally known to those skilled in the art or can be readily obtained from the literature. Exemplary reaction conditions sufficient for performing the chemical transformations provided herein can be found throughout, and in particular, the examples below. It is also contemplated that the reaction conditions can include reagents in addition to those listed in the specific reaction.

The term “contacting” or “contact” refers to the process of bringing into contact at least two distinct species such that they can interact with each other, such as in a non-covalent or covalent binding interaction or binding reaction. It should be appreciated, however, that the resulting complex or reaction product can be produced directly from an interaction or a reaction between the added reagents or from an intermediate from one or more of the added reagents or moieties, which can be produced in the contacting mixture.

### Abbreviations

| Abbreviation | Meaning |
|---|---|
| a/a | Area/area |
| MeCN or CH₃CN | Acetonitrile |
| NH₄OH | Ammonium hydroxide |
| aq. | Aqueous |
| BSA | Benzenesulfonic acid |
| ° C. | Degree Celsius |
| CDCl₃ | Deuterated chloroform |
| DMSO-d₆ | Deuterated DMSO |
| DIPEA | Diisopropylethylamine |
| DMAc | Dimethyl acetamide |
| DMSO | Dimethyl sulfoxide |
| DMF | Dimethylformamide |
| equiv. | Equivalent |
| EtOH | Ethanol |
| EtOAc | Ethyl acetate |
| EtNH₂ | Ethylamine |
| g | Gram |
| HPLC | High-performance liquid chromatography |
| HBr | Hydrobromic acid |
| HCl | Hydrochloric acid |
| H₂ | Hydrogen gas |
| iPAc | Isopropyl acetate |
| i-PrOH | Isopropyl alcohol (isopropanol) |
| kg | Kilogram |
| LDA | Lithium diisopropylamine |
| LED | Light emitting diode |
| LiHMDS | Lithium hexamethyldisilazide |
| MHz | Megahertz |
| MeOH | Methanol |
| Me | Methyl (CH₃) |
| MSA | Methanesulfonic acid |
| MTBE | Methyl tert-tert butyl ether |
| M | Molar |
| n-BuLi | n-butyllithium |
| NMP | N-methyl-2-pyrrolidone |
| Pd(OH)₂/C | Palladium hydroxide on carbon |
| PTSA | para-toluenesulfonic acid |
| POCl₃ | Phosphorus oxychloride |
| KOAc | Potassium acetate |
| K₂CO₃ | Potassium carbonate |
| KOt-Amyl | Potassium tert-amylate |
| KOt-Bu or BuOK | Potassium tert-butoxide |
| ¹H NMR | Proton Nuclear Magnetic Resonance |
| NaHCO₃ | Sodium bicarbonate |
| NaCl | Sodium chloride |
| NaOH | Sodium hydroxide |
| NaOt-Bu | Sodium tert-butoxide |
| H₂SO₄ | Sulfuric acid |
| Red. | Reduction |
| Boc | Tert-butyloxycarbonyl |
| TEA | Triethylamine |
| THF | Tetrahydrofuran |
| TFA | Trifluoroacetic acid |
| TMS | Trimethylsilyl |
| Wt. | Weight |

### Processes

Starting materials and reagents for the preparation of compounds of the present disclosure are generally available from commercial sources or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database).

The following illustrative Schemes 1-4 are directed to certain chemical reactions, processes, methodology for the synthesis of compounds of the present disclosure, as well as certain reagents and novel intermediates.

In Scheme 1, Routes A and B to compound I are depicted. Route B was found to solve deficiencies of Route A with respect to yield and purity. The reaction of A-1 with amine II required higher temperatures that resulted in increased formation of side products and required multiple recrystallizations to purify compound I, providing compound I in low-to-moderate yield, typically 45% yield at manufacturing scale. In contrast, compound I can be prepared with Route B in over 60% overall yield from amine II at manufacturing scale, with purity routinely at 100% (no impurities detected) with a single crystallization.

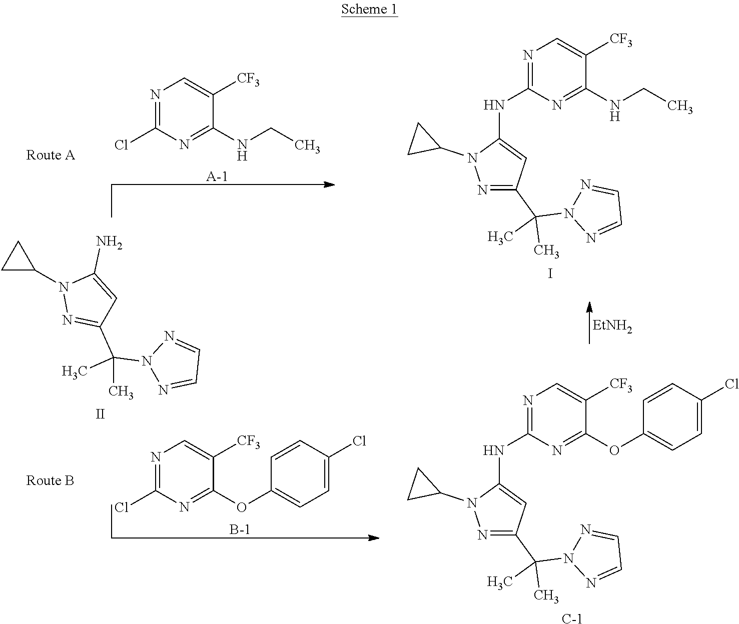

As shown in Scheme 2, other intermediates 2-3 can be used in addition to B-1, where R¹, R², R³, R⁴, and R⁵ are independently H, cyano, halo, methyl, or NO₂. These intermediates can be prepared by coupling phenol 2-1 with dichloride 2-2 in the presence of base. A convenient in situ preparation of dichloride 2-2 was also developed, wherein 2,4-dihydroxy-5-(trifluoromethyl)pyrimidine is treated with POCl₃ and an amine base, thus avoiding the need to isolate compound 2-2 which has a pungent odor and is a strong lachrymator.

When 2-1 is 4-chlorophenol, a mixture of regioisomers was formed favoring 2-chloro-4-(4-chloro-phenoxy)-5-(trifluoromethyl)pyrimidine, a compound of Formula 2-3. It was surprisingly found that the side products such as the unwanted regioisomer could be completely removed through simple crystallization using heptane to isolate B-1 in high purity on manufacturing scale (see Examples 4 and 5a). This regioselective crystallization was also seen with isopropyl alcohol or with mixtures of isopropyl alcohol and water (see Example 5b-c). In many other solvents compound B-1 was typically highly soluble or the selective solubility favoring crystallization of B-1 was not as significant.

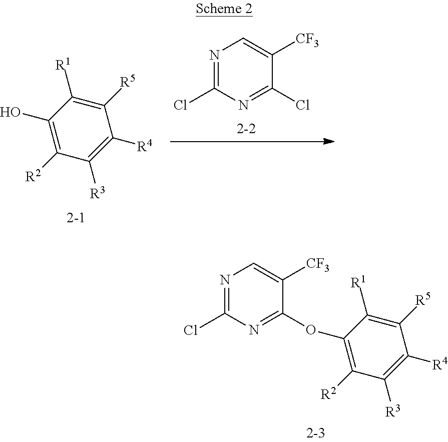

The formation of amine II from ketone IV is shown in Scheme 3. Use of di-Boc compound III (Route B) was found to be superior to use of the hygroscopic and less stable hydrazine salt 3-1 (Route A). Compound III can be prepared from the reaction of di-tert-butylazodicarboxylate with cyclopropylmagnesium bromide or cyclopropanecarboxylic acid (e.g. in presence of cerium trichloride, tetrabutylammonium chloride, cesium carbonate, and 455 nm LED). Boc deprotection of III followed by condensation with ketone IV and subsequent cyclization can be performed as a single pot process in the presence of acid to give aminopyrazole II.

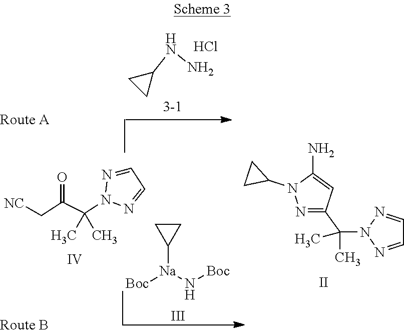

Ketone IV can be prepared as shown in Schemes 4 and 5. Coupling of triazole 4-1 with bromide 4-2 in the presence of a base gives the N²-triazole 4-3 along with significant amounts of the N¹-triazole regioisomer 4-4. Surprisingly it was discovered that the unwanted regioisomer 4-4 can be removed by aqueous washes to give 4-3 in greater than 95% regioisomeric purity. Treatment of 4-3 with a base and acetonitrile gives ketone IV.

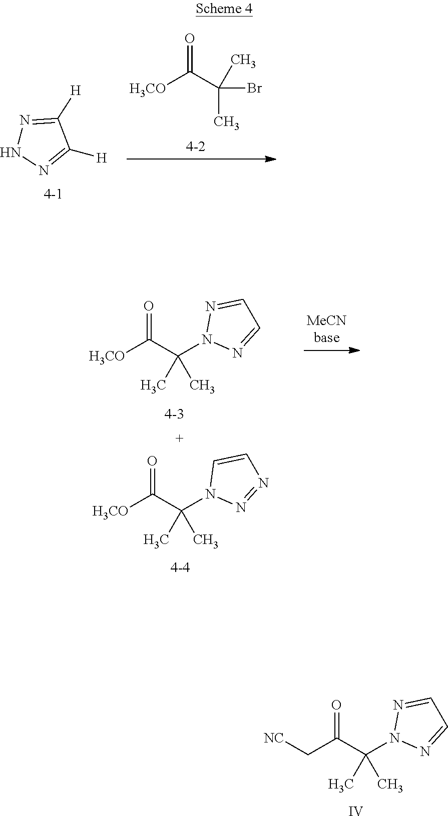

Ketone IV can also be prepared as shown in Scheme 5. A compound of Formula VIII can be coupled with a compound of Formula VI wherein R⁶ is alkyl and R⁷ and R⁸ are independently Br or trimethylsilyl (TMS) in the presence of base. Intermediate IX does not need to be isolated and can be treated with the appropriate reagents and conditions to remove the R⁷ and R⁸ groups to give VII. Ester VII also does not need to be isolated and can be treated with a base and acetonitrile to give ketone IV.

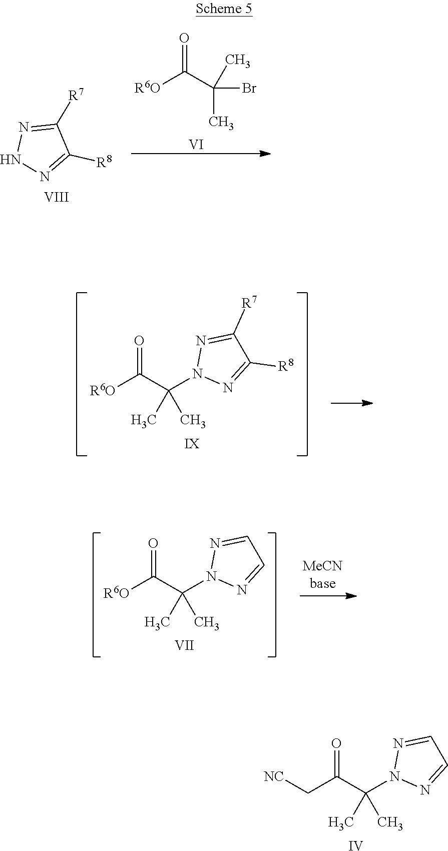

### EXAMPLES

Compounds were characterized and structures confirmed by NMR. The samples for NMR analysis were prepared by complete dissolution of an appropriate amount of material in deuterated solvent (CDCl₃). ¹H NMR spectra were recorded at room temperature at 400 MHz using a Bruker 400 MHz NMR spectrometer.

### Example 1. 2-Cyclopropyl-5-[1-methyl-1-(triazol-2-yl)ethyl]pyrazol-3-amine

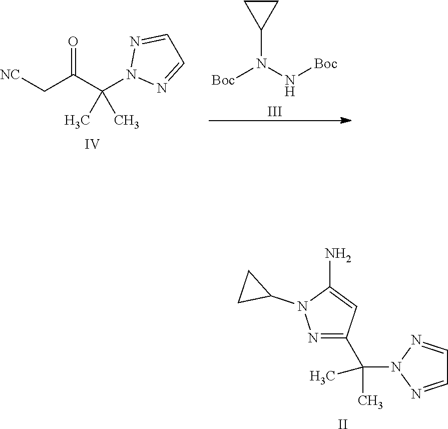

A reactor was charged with ketone IV (1.0 equiv., 45 kg scale), di-Boc III (1.5 equiv.), and ethanol (10 volumes). The reaction mixture was cooled to 0° C. before concentrated H₂SO₄ (1.8 equiv.) was charged to the reactor while maintaining the internal temperature below 10° C. The reaction mixture was agitated at 55° C. for 32 hours. The reaction mixture was cooled to 0° C. before water (4 volumes) was charged to the reactor while maintaining the internal temperature below 10° C. The pH was of the reaction mixture was adjusted to pH 9 with a solution of 28 wt. % aqueous NH₄OH while maintaining the internal temperature below 10° C. The mixture was extracted twice with iPAc (10 volumes and 5 volumes) and the combined organic layers were continuously concentrated under reduced pressure twice with i-PrOH (2 to 3 volumes). The mixture was heated to 45° C. and n-heptane (2 volumes) was charged over 30 minutes. The mixture was cooled to 20° C. over 1 hour and the slurry was agitated for 2 hours at 20° C. The slurry was filtered, the filter cake was washed with n-heptane (3 volumes), and the solids were dried to afford aminopyrazole II in 70% yield and in 99.4% a/a. ¹H NMR (400 MHz, CDCl₃) δ 7.59 (s, 2H), 5.01 (s, 1H), 3.71 (s, 2H), 3.12-3.06 (m, 1H), 2.02 (s, 7H), 1.16-1.06 (m, 1H), 1.02-0.96 (m, 2H).

### Example 2. N-[1-cyclopropyl-3-[1-methyl-1-(2H-1,2,3-triazol-2-yl)ethyl]-1H-pyrazol-5-yl]-4-(4-chlorophenoxy)-5-trifluoromethyl-2-pyrimidinamine

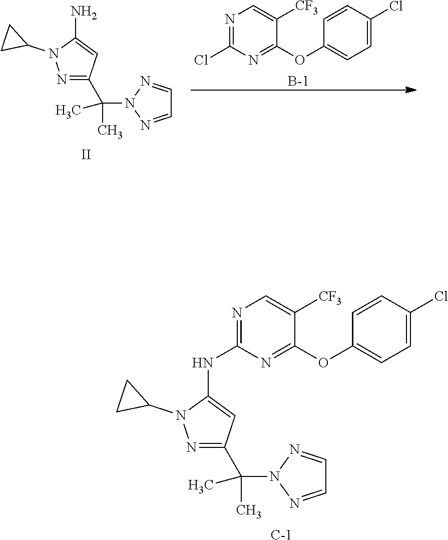

A reactor was charged with aminopyrazole II (1.0 equiv., 40 kg scale), chloropyrimidine B-1 (1.1 equiv.), and NMP (3 volumes). 2,6-lutidine (1.1 equiv.) was charged to the reactor and the reaction mixture was agitated at 67° C. for 48 hours. The reaction mixture was cooled to 27° C. before MTBE (10 volumes) and a 0.25 N aqueous solution of HCl (10 volumes) were charged to the reactor. The organic layer was washed with 0.25 N aqueous solution of HCl (5 volumes) and water (5 volumes). The organic layer was concentrated to 3 volumes and i-PrOH (3 volumes) was charged to the reactor. The mixture was concentrated to 4 volumes and i-PrOH (3 volumes) was charged to the reactor. The mixture was heated to 50° C. and agitated for 3 hours. The mixture was slowly cooled to 20° C. over 3 hours and agitated for 1 hour at 20° C. The slurry was filtered, the filter cake was washed with cold i-PrOH (3 volumes), and the solids were dried to afford C-1 in 74% yield and in 99.9% a/a. ¹H NMR (400 MHz, CDCl₃) δ 8.52 (s, 1H), 7.60 (s, 2H), 7.37 (d, J=8.9 Hz, 2H), 7.10 (d, J=8.9 Hz, 2H), 5.74 (s, 1H), 3.16 (m, 1H), 1.93 (s, 6H), 1.16-1.03 (m, 4H).

### Example 3. N2-(3-(2-(2H-1,2,3-triazol-2-yl)propan-2-yl)-1-cyclopropyl-1H-pyrazol-5-yl)-N4-ethyl-5-(trifluoromethyl)pyrimidine-2,4-diamine

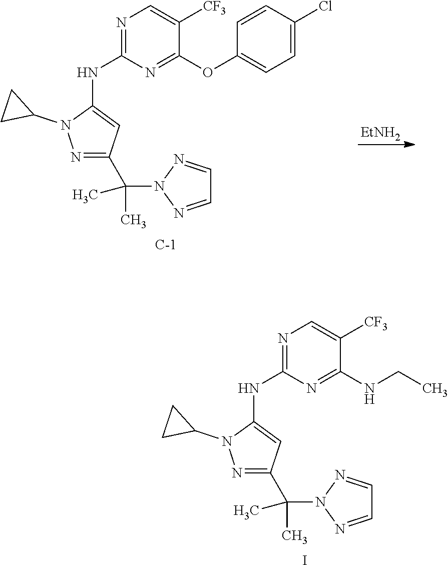

A reactor was charged with C-1 (1.0 equiv., 60 kg scale) and THE (3 volumes). A 70 wt. % aqueous solution of ethylamine (6.0 equiv.) was charged to the reactor and the reaction mixture was agitated at 25° C. for 24 hours. The reaction mixture was concentrated under reduced pressure to 1.5 volumes and MTBE (10 volumes) was charged to the reactor. The organic layer was washed thrice with a 3 wt. % aqueous solution of NaOH (5 volumes) and twice with water (5 volumes). The organic layer was concentrated under reduced pressure to 3 volumes and MTBE (5 volumes) was charged to the reactor. The mixture was heated to 50° C. before n-heptane (2 volumes) was charged to the reactor. The mixture was slowly cooled to −5° C. over 6 hours and agitated at −5° C. for 2 hours. The slurry was filtered, the filter cake was washed with a cold 1:3 v/v mixture of MTBE:n-heptane (4 volumes), and the solids were dried to afford product I in 84% yield and in 100% a/a. ¹H NMR (400 MHz, CDCl₃) δ 8.11 (s, 1H), 7.61 (s, 2H), 7.33 (s, 1H), 6.11 (s, 1H), 5.18 (s, 1H), 3.41 (qd, J=7.2, 5.2 Hz, 2H), 3.23 (tt, J=6.9, 3.6 Hz, 1H), 2.09 (s, 6H), 1.31-1.16 (m, 5H), 1.16-1.08 (m, 2H).

### Example 4. 2-Chloro-4-(4-chloro-phenoxy)-5-(trifluoromethyl)pyrimidine

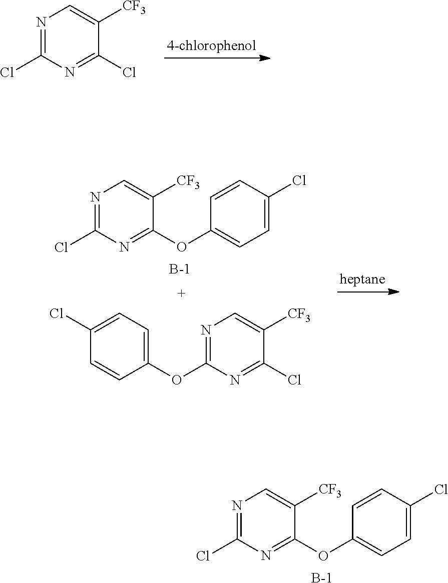

A reactor was charged with 2,4-dichloro-5-(trifluoromethyl)pyrimidine (1.0 equiv., 110 kg scale), potassium carbonate (1.1 equiv.), and acetone (8 volumes). The reaction mixture was cooled to 0° C. before a solution of 4-chlorophenol (1.0 equiv.) in acetone (1 volume) was charged to the reactor while maintaining the internal temperature below 10° C. The reaction mixture was agitated at 5° C. for 40 hours to provide a mixture of B-1 along with its regioisomer. The reaction mixture was filtered and the filter cake was washed twice with acetone (2 volumes). The filtrate was concentrated under reduced pressure to 3 volumes. n-Heptane (5 volumes) was charged to the reactor and the mixture was concentrated under reduced pressure to 3 volumes. n-Heptane (10 volumes) was charged to the reactor and the mixture was concentrated under reduced pressure to 6 volumes. The concentrated mixture was agitated at 60° C. for 2 hours before being cooled to 0° C. over 6 hours. The slurry was agitated at 0° C. for 3 hours. The slurry was filtered (to remove the unwanted regioisomer) and the filter cake was charged back to the reactor with n-heptane (2 volumes). The slurry was agitated at 0° C. for 1 hour. The slurry was filtered, the filter cake was washed with cold n-heptane (0.5 volumes), and the solids were dried to afford B-1 in 58% yield and in 100% a/a. ¹H NMR (400 MHz, CDCl₃) δ 8.72 (d, J=0.9 Hz, 1H), 7.48-7.39 (m, 2H), 7.17-7.11 (m, 2H).

### Example 5. 2-Chloro-4-(4-chloro-phenoxy)-5-(trifluoromethyl)

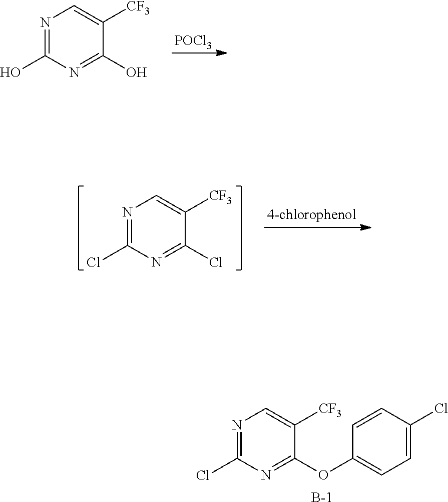

A reactor was charged with 2,4-dihydroxy-5-(trifluoromethyl)pyrimidine (1.0 equiv., 30 kg scale) and acetonitrile (3 volumes). The reaction mixture was adjusted to 20° C. before POCl₃ (3.0 equiv.) was charged to the reactor while maintaining the internal temperature between 2° and 30° C. The reaction mixture was heated to 55° C. before DIPEA (3.0 equiv.) was charged to the reactor over 6 hours. Following the addition, the reaction mixture was agitated at 55° C. for 40 hours. The reaction mixture was adjusted to 20° C. before being charged to a separate reactor, which contained n-heptane (10 volumes) and water (10 volumes). The aqueous layer was isolated and extracted twice with n-heptane (5 volumes). The combined organic layers were washed once with water (5 volumes). Charcoal (5 wt. %) was charged to the combined organic layers and agitated at 25° C. for 2 hours. The slurry was filtered and the filter cake was washed with n-heptane (1 volume). The filtrate containing the n-heptane solution of 2,4-dichloro-5-(trifluoromethyl)pyrimidine (69% assay yield, 97.5% a/a) was used directly in the next step without further purification. ¹H NMR (400 MHz, CDCl₃) δ 8.83 (s, 1H).

### Example 5a. 4-Chlorophenol Coupling and Heptane Crystallization

A reactor was charged with a n-heptane solution of 2,4-dichloro-5-(trifluoromethyl)pyrimidine (1.0 equiv., 37 kg scale). The reaction mixture was cooled to 5° C. A solution of K₂CO₃ (1.1 equiv.) and 4-chlorophenol (1.0 equiv.) in water (5 volumes) was charged to the reactor while maintaining the internal temperature between 0 and 10° C. The reaction mixture was agitated at 5° C. for 39 hours. A solution of K₂CO₃ (0.1 equiv.) and 4-chlorophenol (0.1 equiv.) in water (1 volume) was charged to the reactor while maintaining the internal temperature between 0 and 10° C. The reaction mixture was agitated at 5° C. for 16 hours. The reaction mixture was adjusted to 20° C. before n-heptane (5 volumes) was charged to the reactor. The reaction mixture was agitated for 30 minutes before the organic layer was isolated and washed with water (5 volumes) until pH 7. The organic layer was concentrated under reduced pressure to 6 volumes. The slurry was heated to 80° C. for 2 hours before being adjusted to 60° C. and agitated for 2 hours. The slurry was cooled to 0° C. over 6 hours and agitated for 3 hours. The slurry was filtered, the filter cake was washed with cold n-heptane (2 volumes), and the solids were dried to afford B-1 in 75% yield and in 99.9% a/a. ¹H NMR (400 MHz, CDCl₃) δ 8.72 (d, J=0.9 Hz, 1H), 7.48-7.39 (m, 2H), 7.17-7.11 (m, 2H).

### Example 5b. 4-Chlorophenol Coupling and Heptane Crystallization 4-Chlorophenol Coupling and IPA/Water Crystallization

A reactor was charged with a n-heptane solution of 2,4-dichloro-5-(trifluoromethyl)pyrimidine (1.0 equiv., scaling factor, 50 g scale) and 4-chlorophenol (1.1 equiv.). The reaction mixture was cooled to 5° C. A solution of K₂CO₃ (1.1 equiv.) in water (5 volumes) was charged to the reactor while maintaining the internal temperature between 0 and 10° C. The reaction mixture was agitated at 5° C. for 24 hours. The reaction mixture was then adjusted to 30° C. and n-heptane (4 volumes) was charged to the reactor. The resulting solution was agitated for an additional 40 hours. The organic layer was isolated and washed twice with water (5 volumes) until pH 7. The organic layer was concentrated under reduced pressure to 3 volumes. The slurry was adjusted to 6 volumes with isopropanol and heated to 30° C. Water (2 volumes) was added and the resulting slurry was agitated for 30 minutes, cooled to 0° C. over 3 hours and agitated for an additional two hours. The slurry was filtered and the wet cake was combined with isopropanol (3 volumes) and the temperature was adjusted to 30° C. Water (1 volume) was added at 30° C. and the slurry was cooled to 0° C. over 3 hours and agitated for another 2 hours. The slurry was filtered, the filter cake was washed with cold n-heptane (0.5 volumes), and the solids were dried to afford B-1 in 78% yield and in 99.6% a/a. ¹H NMR (400 MHz, CDCl₃) δ 8.72 (d, J=0.9 Hz, 1H), 7.48-7.39 (m, 2H), 7.17-7.11 (m, 2H).

### Example 5c. 4-Chlorophenol Coupling and Heptane Crystallization 4-Chlorophenol Coupling and IPA Crystallization

A reactor was charged with a n-heptane solution of 2,4-dichloro-5-(trifluoromethyl)pyrimidine (1.0 equiv., scaling factor, 35 g scale) and 4-chlorophenol (1.1 equiv.). The reaction mixture was adjusted to 22° C. A solution of K₂CO₃ (1.1 equiv.) in water (5 volumes) was charged to the reactor while maintaining the internal temperature between 2° and 30° C. The reaction mixture was agitated at 30° C. for 45 hours. The organic layer was isolated and washed twice with water (5 volumes) Isopropanol (3 volumes) was charged and the temperature was adjusted to 50° C. The resulting solution was cooled to 0° C. over five hours and then held for three hours. The slurry was filtered, the filter cake was washed with cold isopropanol (2 volumes), and the solids were dried to afford DN11247 in 68% yield and in 99.9% a/a. ¹H NMR (400 MHz, CDCl₃) δ 8.72 (d, J=0.9 Hz, 1H), 7.48-7.39 (m, 2H), 7.17-7.11 (m, 2H).

### Example 6. Methyl 2-methyl-2-(triazol-2-yl)propanoate

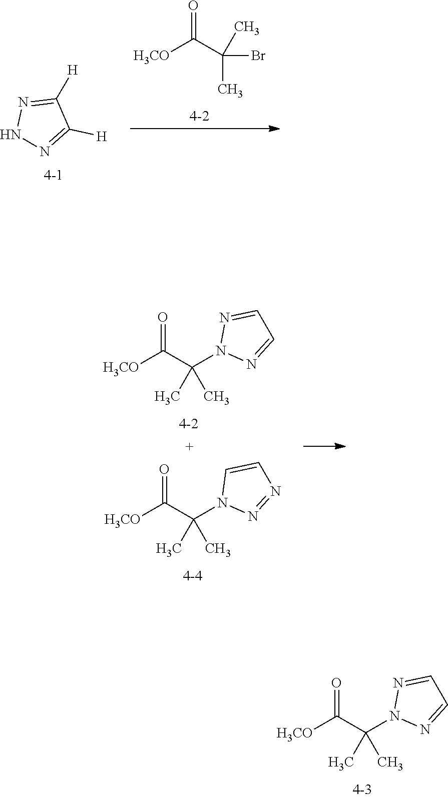

A reactor was charged with NaOt-Bu (1.1 equiv.) and NMP (5 volumes). The reaction mixture was cooled to 5° C. and 2H-1,2,3-triazole 4-1 (1.0 equiv., 100 kg scale) was charged to the reactor while maintaining the internal temperature below 10° C. The reaction mixture was heated to 55° C. and methyl 2-bromoisobutyrate 4-2 (1.1 equiv.) was charged to the reactor while maintaining the internal temperature below 60° C. The reaction mixture was agitated at 55° C. for 18 hours and then cooled to 5° C. to give a mixture of triazole regioisomers 4-3 and 4-4. Water (10 volumes) was charged to the reactor while maintaining the internal temperature below 10° C. The mixture was extracted twice with MTBE (10 volumes) and the combined organic layers were washed twice with 2.5 M aq. HCl (3 volumes) to remove the unwanted regioisomer. The combined organic layers were washed with 5 wt. % aq. NaHCO₃ (3 volumes) and water (3 volumes). The combined organic layers were concentrated under reduced pressure to 2 volumes and MTBE (4 volumes) was charged to the reactor. The mixture was concentrated under reduced pressure to 2 volumes and MTBE (4 volumes) was charged to the reactor. The mixture was concentrated under reduced pressure to 2 volumes and MTBE (4 volumes) was charged to the reactor to afford N²-triazole 4-3 (32% assay yield, 84.9% a/a) as a solution in MTBE, which was either exchanged to THE or used directly in the preparation of ketone IV. ¹H NMR (400 MHz, CDCl₃) δ 7.64 (s, 2H), 3.69 (s, 3H), 1.95 (s, 6H).

### Example 7a. 4-Methyl-3-oxo-4-(triazol-2-yl)pentanenitrile

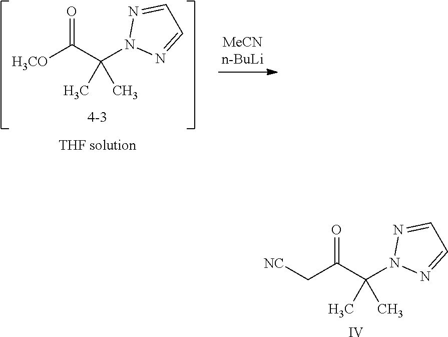

A reactor was charged with CH₃CN (2.1 equiv.) and THE (10 volumes). The reaction mixture was cooled to −75° C. before a 2.5 M solution of n-butyllithium in hexane (2.0 equiv.) was charged to the reactor while maintaining the internal temperature below −70° C. The reaction mixture was agitated at −75° C. for 1 hour. A solution of 4-3 (1.0 equiv., 113 kg scale) in THE (4 volumes) was charged to the reactor while maintaining the internal temperature below −70° C. The reaction mixture was agitated at −75° C. for 2.5 hours. Water (5 volumes) was charged to the reactor while maintaining the internal temperature below 5° C. and the reaction mixture was warmed to 5° C. The pH of the mixture was adjusted to 3 to 5 with 3 M aqueous HCl. The aqueous layer was isolated and extracted twice with EtOAc (5 volumes). The combined organic layers were washed with saturated aqueous NaCl (5 volumes) and concentrated under reduced pressure to 1.5 volumes. The mixture was continuously concentrated 4 times with MTBE (3 volumes) to 3 volumes. The slurry was agitated at 20° C. for 2 hours. The slurry was filtered, the filter cake was washed with MTBE (1 volume), and the solids were dried to afford ketone IV in 60% yield and in 100% a/a. ¹H NMR (400 MHz, CDCl₃) δ 7.74 (s, 2H), 3.10 (s, 2H), 1.88 (s, 6H).

### Example 7b. 4-Methyl-3-oxo-4-(triazol-2-yl)pentanenitrile

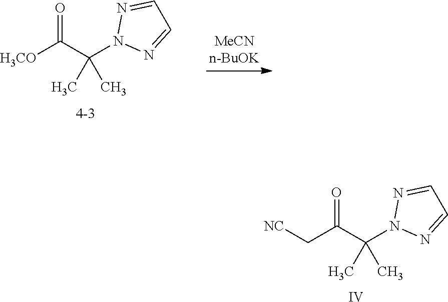

A reactor was charged with 4-3 (1.0 equiv., 10 g scale), CH₃CN (2.1 equiv.), and THF (3 volumes). The reaction mixture was cooled to −10° C. before a 1.0 M solution of KOt-Bu in THE (2.0 equiv.) was charged to the reactor while maintaining the internal temperature below 0° C. The reaction mixture was agitated at −10° C. for 12 hours. Water (5 volumes) was charged to the reactor while maintaining the internal temperature below 5° C. and the reaction mixture was warmed to 5° C. The pH of the mixture was adjusted to 3 to 5 with 3 M aqueous HCl. The aqueous layer was isolated and extracted twice with EtOAc (5 volumes). The combined organic layers were washed with saturated aqueous NaCl (5 volumes) and concentrated under reduced pressure to 1.5 volumes. The mixture was continuously concentrated 4 times with MTBE (3 volumes) to 3 volumes. The slurry was agitated at 20° C. for 2 hours. The slurry was filtered, the filter cake was washed with MTBE (1 volume), and the solids were dried to afford ketone IV in 86% yield and in 100% a/a. ¹H NMR (400 MHz, CDCl₃) δ 7.74 (s, 2H), 3.10 (s, 2H), 1.88 (s, 6H).

### Example 7c. 4-Methyl-3-oxo-4-(triazol-2-yl)pentanenitrile

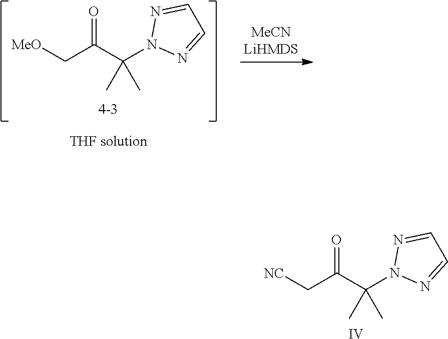

A reactor was charged with a solution of 4-3 (1.0 equiv., scaling factor, 37 kg scale) in THE (6 volumes). Acetonitrile (2.8 equiv.) was charged and the resulting solution was cooled to −10° C. A 1.0 M solution of lithium bis(trimethylsilyl)amide in THE (2.5 equiv.) was charged to the reactor while maintaining the internal temperature below 10° C. The reaction was agitated at 20° C. for 2 hours. The temperature was adjusted to 0° C. and water (5 volumes) was charged to the reactor while maintaining the internal temperature below 10° C. The pH of the mixture was adjusted to 3 to 4 with 6 M aqueous HCl. The aqueous layer was isolated and extracted with MTBE (5 volumes). The combined organic layers were washed with a solution of 10 wt % aqueous NaCl (5 volumes) and concentrated under reduced pressure to 1.5 volumes. The mixture was continuously concentrated two times with MTBE (3 volumes) to 1.5 volumes. MTBE (1 volume) was charged and the temperature was adjusted to 50° C. The resulting solution was cooled to 5° C. over two hours and then held for three hours. The slurry was filtered, the filter cake was washed with cold MTBE (1 volume), and the solids were dried to afford IV in 83% yield and in 99.9% a/a. ¹H NMR (400 MHz, CDCl3) δ 7.74 (s, 2H), 3.10 (s, 2H), 1.88 (s, 6H).

### Example 8. 4-Methyl-3-oxo-4-(triazol-2-yl)pentanenitrile

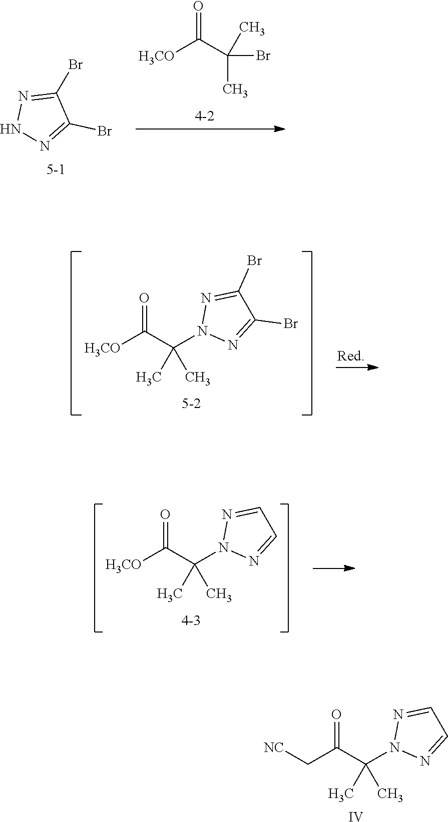

A reactor was charged with dibromotriazole 5-1 (1.0 equiv., 24 kg scale), potassium carbonate (1.0 equiv.), and NMP (5 volumes). Methyl 2-bromoisobutyrate (1.3 equiv.) was charged to the reactor while maintaining the internal temperature below 20° C. The reaction mixture was agitated at 35° C. for 24 hours. The reaction mixture was cooled to 10° C. before 3.2 wt. % aqueous HCl (15 volumes) was added while maintaining the internal temperature below 20° C. The mixture was extracted twice with MTBE (10 volumes) and the combined organic layers were washed with water (5 volumes). The combined organic layers were concentrated under reduced pressure to 2 volumes. MeOH (2 volumes) was charged to the reactor and the mixture was concentrated under reduced pressure to 2 volumes. MeOH (6 volumes) and water (0.7 volumes) was charged to the reactor to afford methyl ester 5-2 (90% assay yield, 86.3% a/a) as a solution in MeOH and water which was used directly in the preparation of 4-3. ¹H NMR (400 MHz, CDCl₃) δ 3.70 (s, 3H), 1.91 (s, 6H).

A reactor was charged with 5-2 (1.0 equiv., 15 kg scale) as a solution in MeOH and water (7 volumes), 20 wt. % Pd(OH)₂/C (0.015 w/w, dry basis), and KOAc (3.0 equiv.). The mixture was pressurized with H₂ to 1.5 mPa and agitated at 55° C. for 10 hours. The reaction mixture was filtered and the filter cake was washed with MeOH (2 volumes). The filtrate was concentrated under reduced pressure to 1 volume. MTBE (10 volumes) and 8 wt. % aqueous NaHCO₃ (8 volumes) were charged to the reactor. The organic layer was isolated and washed with water (5 volumes). The combined aqueous layers were extracted with MTBE (5 volumes) and the combined organic layers were concentrated under reduced pressure to 1 volume. THF (3 volumes) was charged to the reactor and the mixture was concentrated under reduced pressure to 1 volume. THE (3 volumes) was charged to the reactor and the mixture was concentrated under reduced pressure to 1 volume. THE (1.5 volumes) was charged to the reactor to afford 4-3 (97% assay yield, 98.4% a/a) as a solution in THE which was used directly in the preparation of IV. ¹H NMR (400 MHz, CDCl₃) δ 7.64 (s, 2H), 3.69 (s, 3H), 1.95 (s, 6H).

A reactor was charged with CH₃CN (2.1 equiv.) and THE (10 volumes). The reaction mixture was cooled to −75° C. before a 2.5 M solution of n-butyllithium in hexane (2.0 equiv.) was charged to the reactor while maintaining the internal temperature below −70° C. The reaction mixture was agitated at −75° C. for 1 hour. A solution of 4-3 (1.0 equiv., 113 kg scale) in THE (4 volumes) was charged to the reactor while maintaining the internal temperature below −70° C. The reaction mixture was agitated at −75° C. for 2.5 hours. Water (5 volumes) was charged to the reactor while maintaining the internal temperature below 5° C. and the reaction mixture was warmed to 5° C. The pH of the mixture was adjusted to 3 to 5 with 3 M aqueous HCl. The aqueous layer was isolated and extracted twice with EtOAc (5 volumes). The combined organic layers were washed with saturated aqueous NaCl (5 volumes) and concentrated under reduced pressure to 1.5 volumes. The mixture was continuously concentrated 4 times with MTBE (3 volumes) to 3 volumes. The slurry was agitated at 20° C. for 2 hours. The slurry was filtered, the filter cake was washed with MTBE (1 volume), and the solids were dried to afford IV in 60% yield and in 100% a/a. ¹H NMR (400 MHz, CDCl₃) δ 7.74 (s, 2H), 3.10 (s, 2H), 1.88 (s, 6H).

What is claimed is:

1. A compound of Formula B: 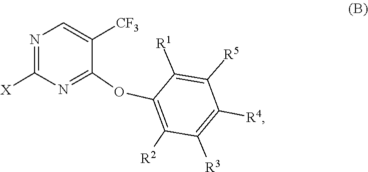
or a salt thereof, wherein:
X is chloro or 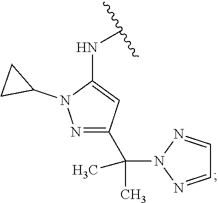 [SMILES: *Nc1cc(C(C)(C)n2nccn2)nn1C1CC1]
and
R¹, R², R³, R⁴, and R⁵ are independently H, cyano, halo, methyl, or NO₂, provided that
a) when X is Cl and R¹, R², R³, and R⁵ are H, then R⁴ is not NO₂ or H;
b) when X is Cl and R¹ and R² are CH₃, then R⁴ is not cyano; and
c) when X is Cl, then R³ is not NO₂.

2. The compound of claim 1 that is 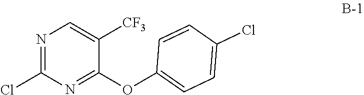 [SMILES: FC(F)(F)c1cnc(Cl)nc1Oc1ccc(Cl)cc1]
or a salt thereof.

3. The compound of claim 1 that is 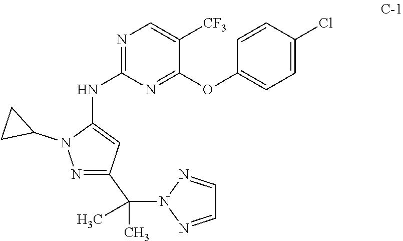 [SMILES: CC(C)(c1cc(Nc2ncc(C(F)(F)F)c(Oc3ccc(Cl)cc3)n2)n(C2CC2)n1)n1nccn1]
or a salt thereof.

4. A method for preparing compound I or a salt thereof 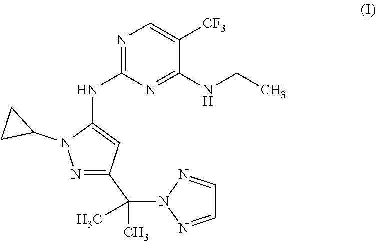 [SMILES: CCNc1nc(Nc2cc(C(C)(C)n3nccn3)nn2C2CC2)ncc1C(F)(F)F]
comprising:
a) contacting compound II with a compound Formula B and a base to produce a compound of Formula C 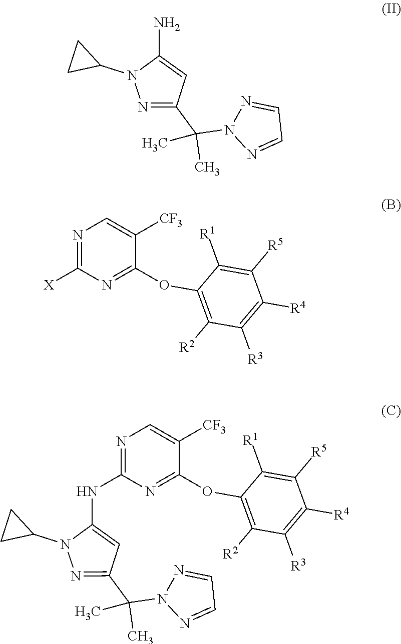
wherein R¹, R², R³, R⁴, and R⁵ are independently H, cyano, halo, methyl, or NO₂; and
b) contacting a compound of Formula C with ethylamine;
to produce compound I.

5. The method of claim 4, wherein the compound of Formula B is compound B-1 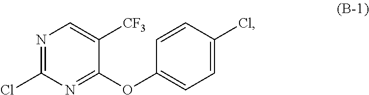 [SMILES: FC(F)(F)c1cnc(Cl)nc1Oc1ccc(Cl)cc1]
and the compound of Formula C is compound C-1

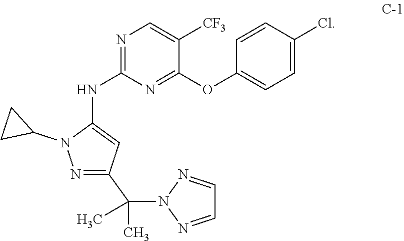 [SMILES: CC(C)(c1cc(Nc2ncc(C(F)(F)F)c(Oc3ccc(Cl)cc3)n2)n(C2CC2)n1)n1nccn1]

6. The method of claim 5, wherein the compound C-1 is produced in 96% or greater, 97% or greater, 98% or greater, or 99% or greater regioisomeric purity.

7. The method of claim 5, wherein the base is 2,6-lutidine or 2,4,6-collidine and reaction is done in NMP or DMSO at a temperature from about 60° C. to about 70° C.

8. The method of claim 5, wherein the compound B-1 is prepared by
a) contacting compound D-1 with compound D-2 and a base to the produce the compound B-1; and 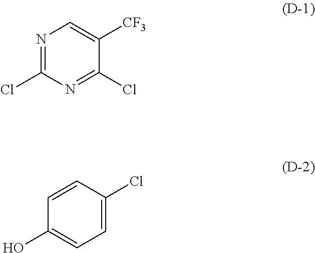 [SMILES: FC(F)(F)c1cnc(Cl)nc1Cl ; Oc1ccc(Cl)cc1]
b) optionally crystallizing the compound B-1 from heptane, isopropanol, or an isopropanol/water mixture.

9. The method of claim 8, wherein the compound B-1 is crystallized in 90% or greater, 95% or greater, or 99% or greater regioisomeric purity.

10. The method of claim 8, wherein the base is K₂CO₃ or NaOH.

11. The method of claim 10, wherein the base is K₂CO₃.

12. The method of claim 8, wherein the compound D-1 is prepared in situ by contacting compound E-1 with POCl₃ 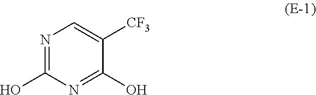 [SMILES: Oc1ncc(C(F)(F)F)c(O)n1]
to produce the compound D-1.

13. The method of claim 12, wherein the compound E-1 is contacted with POCl₃ and diisopropylethylamine.

14. The method of claim 4, wherein the compound I is obtained in greater than 98% or greater than 99% purity.

15. The method of claim 14, wherein the compound I is obtained in greater than 99.5% purity.

16. The method of claim 4, wherein the compound II is prepared by contacting compound III with compound IV and an acid

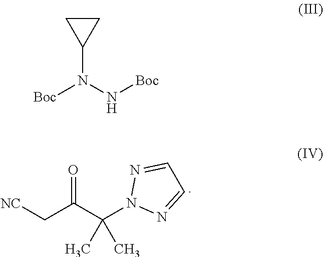 [SMILES: CC(C)(C)OC(=O)NN(C(=O)OC(C)(C)C)C1CC1 ; CC(C)(C(=O)CC#N)n1nccn1]

17. The method of claim 16, wherein the acid is H₂SO₄ or HCl in an alcoholic solvent.

18. The method of claim 16, wherein compound III is contacted with compound IV at a temperature from about 50° C. to about 60° C.

19. The method of claim 16, wherein the compound IV is prepared by
a) contacting compound V with a compound of Formula VI and a first base to produce a compound of Formula VII 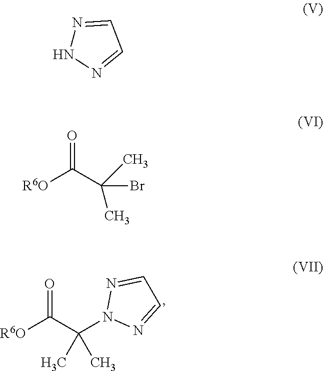
wherein R⁶ is alkyl;
b) washing the compound of Formula VII with an aqueous solution to remove N¹-triazole regioisomer and obtain the compound of Formula VII in 95% or greater regioisomeric purity; and
c) contacting the compound of Formula VII with a second base and CH₃CN;
under conditions sufficient to produce the compound IV.

20. The method of claim 19, wherein R⁶ is methyl and the compound of Formula VII is VII-1 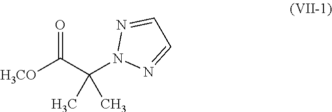
having 95% or greater regioisomeric purity.

21. The method of claims 19 or 20, wherein the compound of Formula VII is obtained in 98% or greater regioisomeric purity.

22. The method of claims 19 or 20, wherein the compound of Formula VII is washed with an aqueous solution at least twice.

23. The method of claim 22, wherein the aqueous solution is an acidic aqueous solution.

24. The method of claim 23, wherein the aqueous solution is aqueous HCl.

25. The method of claim 19, wherein the first base is an alkoxide base.

26. The method of claim 25, wherein the first base is NaOt-Bu.

27. The method of claim 19, wherein the second base is n-BuLi, KOt-Bu, LiHMIDS, LDA, NaOt-Bu, or Kot-Amyl.

28. The method of claim 27, wherein the second base is n-BuLi or LiHMIDS.

29. The method of claim 19, wherein the compound IV is obtained in at least 99% or greater purity.

30. The method of claim 16, wherein the compound IV is prepared by
a) contacting a compound of formula VIII 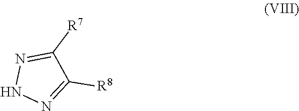
with a compound of Formula VI and a first base to produce a compound of Formula IX 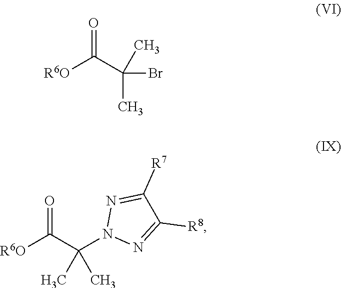
wherein R⁶ is alkyl and R⁷ and R⁸ are independently Br or trimethylsilyl (TMS);
b) contacting the compound of Formula IX when at least one of R⁷ and R⁸ is Br with H₂ or HCO₂H and a hydrogenation catalyst;
and/or contacting the compound of Formula IX when at least one of R⁷ and R⁸ is TMS with base;
to produce a compound of Formula VII 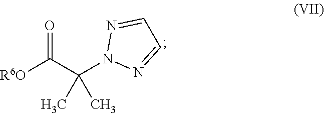
and
c) contacting a compound of Formula VII with a second base and CH₃CN;
to produce the compound IV.

31. The method of claim 30, wherein R⁶ is CH₃ and R⁷ is Br.

32. The method of claims 30 or 31, wherein the hydrogenation catalyst is a Pd catalyst.

33. The method of claims 30 or 31, wherein the first base is an inorganic base.

34. The method of claim 33, wherein the first base is K₂CO₃.

35. The method of claims 30 or 31, wherein the second base is n-BuLi, Kot-Bu, LiHMDS, LDA, NaOt-Bu, or Kot-Amyl.

36. The method of claim 35, wherein the second base is n-BuLi or LiHIMIDS.

37. A compound of Formula IX 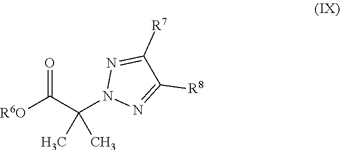
or a salt thereof, wherein:
R⁶ is alkyl and R⁷ and R⁸ are independently Br or trimethylsilyl (TMS).

38. The compound of claim 37 that is 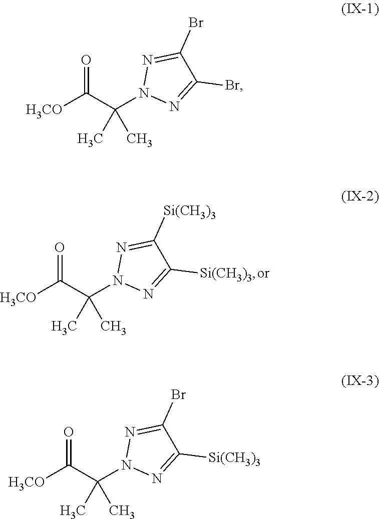
or a salt thereof.
